(19) United States
(12) Patent Application Publication
(10) Pub. No.: US 2010/0055196 A1
(43) Pub. Date: Mar. 4, 2010

(54) SANITIZING COMPOSITION

(75) Inventor: Keith Martin MacGregor, West Yorkshire (GB)

Correspondence Address: CONNOLLY BOVE LODGE & HUTZ LLP, 1875 EYE STREET, N.W., SUITE 1100, WASHINGTON, DC, 20006, US

(73) Assignee: Ebiox,Limited, Warrington (GB)

(21) Appl. No.: 11/571,148

(22) PCT Filed: Jun. 20, 2005
(86) PCT No.: PCT/GB05/02430

§ 371 Date: Nov. 10, 2009

(30) Foreign Application Priority Data
Jun. 25, 2004 (GB) 0414244.4

(51) Int. Cl. A01N 59/00

(52) U.S. Cl. 424/600

(57) ABSTRACT

A sanitizing composition capable of destroying pathogens present at a locus and/or of removing biofilm present at a locus (for example MRSA or Legionella) aqueous solution: at least one surfactant, preferably non-ionic; at least one antimicrobial agent, preferably a biguanide and/or quaternary ammonium compound; at least one acid, preferably organic; and chlorine dioxide. The sanitizing composition is formed by making two precursor compositions, one containing the chlorine dioxide stabilised in an alkaline medium, and the other containing acid. These precursor compositions are mixed to form a concentrate composition. A dwell time is allowed. Once the dwell time has elapsed the concentrate composition is diluted with water, and the resulting sanitizing composition can be used.

The present invention relates to a sanitizing composition, particularly to a sanitizing composition for use in medical facilities.

A known problem in medical facilities is the spread of pathogens. These pathogens—typically bacteria—may stem from only one patient, or region, in such a facility but if not quickly eradicated may spread to contaminate other patients, or regions. One known phenomenon which assists pathogens in spreading is the formation of a biofilm.

Biofilms form when micro-organisms adhere to a surface. They grow and become a culture medium for more micro-organisms. A biofilm can be formed by a single species or micro-organism, for example a bacterium, fungus, algae, or protozoa. However, biofilms may often be formed by multiple species of micro-organism; for example they may often be formed of multiple species of bacteria. Alternatively or additionally they may be formed from debris. The debris may be from living organisms, for example sebum or dead skin cells. Alternatively or additionally the debris may be from inanimate sources, for example corrosion products.

The formation of biofilms is a serious problem with grave consequences in medical facilities where, inevitably, harmful pathogens such as Methicillin Resistant Staphylococcus aureus (MRSA), Legionella (responsible for legionnaires disease) and Escherichia coli (E. coli) may be present. The need to develop technologies to stop these bacteria from spreading is paramount.

Accordingly, there is a continued need for a means of combating pathogens in medical facilities. In particular there is a need to destroy and prevent the formation of biofilms in which the pathogens may flourish.

According to a first aspect of the present invention there is provided a sanitizing composition capable of destroying pathogens present at a locus and/or of removing biofilm present at a locus, the composition comprising, in aqueous solution:

at least one surfactant, preferably present in an amount in the range 0.05-5% w/w;

at least one antimicrobial agent, preferably present in an amount in the range 0.003-4% w/w;

at least one acid, preferably present in an amount in the range 0.1-5%;

and, optionally, chlorine dioxide, preferably present in an amount in the range 0.001-4% w/w.

Preferably, the at least one surfactant is present in an amount in the range 0.1-2% w/w, more preferably in an amount in the range 0.2-1% w/w, most preferably in an amount in the range 0.3-0.6% w/w. In a particularly preferred embodiment, the at least one surfactant is present at a concentration of about 0.45-0.55% w/w.

Preferably the surfactant is a non-ionic surfactant.

Examples of suitable non-ionic surfactants include but are not restricted to: alkoxylated alcohols including ethoxylated and propoxylated fatty alcohols as well as ethoxylated and propoxylated alkyl phenols, preferably having alkyl groups with carbon chain length of from 7 to 16, more preferably 8 to 13.

A preferred group of non-ionic surfactants is alkylphenol ethoxylates (APEs). The structure of suitable alkylphenol ethoxylates usually comprises an alkylphenol with a side chain of several ethoxylate groups. The alkyl group is lo typically a branched nonyl-, octyl- or dodecyl-chain. Examples of suitable alkylphenol ethoxylates include, but are not restricted to: poly (oxy-1,2-ethanol) alpha-(nonylphenol)-omega-hydroxy; Ultranex NP95; Surfonic N95; Polytergent B300; Tergitol NP9; Synperonic NP9.5; Marlophen 89.5. A particularly preferred non-ionic surfactant is a nonylphenol ethoxylate, especially that sold under the trade mark Synperonic N.

Preferably, the at least one antimicrobial agent is present in an amount in the range 0.01-2% w/w, more preferably in an amount in the range 0.05-1% w/w, most preferably in an amount in the range 0.1-0.6% w/w. In a particularly preferred embodiment, the at least one antimicrobial agent is present in an amount in the range 0.2-0.5% w/w, and most preferably it is at a concentration of about 0.3-0.4% w/w.

Preferably, the antimicrobial agent comprises a quaternary ammonium compound. Preferably, such an antimicrobial agent has the following general formula:

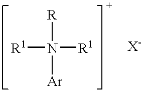

where Ar is an optionally substituted aryl or heteroaryl group, R is any C6 or above unsubstituted branched or linear alkyl group, each group R1 is independently selected from any C1 to C4 branched or unbranched unsubstituted alkyl, and X is a halide anion.

Optional substituents of an aryl or heteroaryl group Ar include halo, cyano, and C1-C4 alkoxy and C1-C4 haloalkyl groups. There may suitably be 1-3 substituents. Preferably, however, Ar is an unsubstituted aryl or heteroaryl group.

Preferably, Ar is selected from optionally substituted phenyl, benzyl, napthyl and pyridyl groups. Most preferably, Ar is an optionally substituted aryl group. Most preferably Ar is an optionally substituted benzyl group.

Preferably, R is any C8 or above unsubstituted branched or linear alkyl group. More preferably, R is any C12 to C20 unsubstituted branched or linear alkyl group. Most preferably, R is any C12 to C20 unsubstituted linear alkyl group. In a particularly preferred embodiment, R is an unbranched unsubstituted C18 alkyl group.

Preferably, R1 are each independently selected from methyl, ethyl, propyl, butyl and isopropyl. More preferably, R1 are each methyl groups.

Preferably, X is a chloride, bromide or iodide anion. Most preferably, X is a chloride anion.

Preferably, the antimicrobial compound comprises benzalkonium chloride (BAC), or may be a derivative thereof.

When a quaternary ammonium compound is present, it is preferably present in an amount in an amount in the range 0.001-3% w/w, more preferably in an amount in the range 0.01-1% w/w, more preferably in an amount in the range 0.05-0.5% w/w, and most preferably in an amount in the range 0.1-0.4% w/w; and especially, at a concentration of about 0.2-0.3% w/w.

Alternatively or additionally, the antimicrobial agent comprises a biguanide compound.

By “biguanide compound” we mean a chemical having the following functional group:

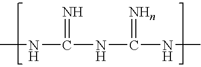

where n=1 or 2.

When n=2, the alpha nitrogen becomes tetravalent and hence positively charged. In this case, the charge may be equalised by any anion, preferably a halide anion such as chloride, bromide or iodide.

Preferably, a biguanide antimicrobial agent is a polymeric biguanide compound. A particularly preferred polymeric biguanide compound is polyhexamethylenebiguanide (PHMB), or derivatives thereof.

When a biguanide antimicrobial agent is present, it is preferably present in an amount in an amount in the range 0.001-2% w/w, more preferably in an amount in the range 0.005-1% w/w, more preferably in an amount in the range 0.01-0.5% w/w, and most preferably in an amount in the range 0.02-0.2% w/w; and especially, in an amount in the range 0.05-0.15% w/w.

Preferably both a quaternary ammonium antimicrobial agent and a biguanide antimicrobial agent are present. Preferably they are present in amounts to satisfy three numerical definitions: at least one of the numerical definition which applies to the quaternary ammonium antimicrobial agent itself; at least one of the numerical definition which applies to the biguanide antimicrobial agent itself; and at least one of the numerical definitions which applies to the “at least one antimicrobial agent”

Preferably the at least one acid is present in an amount in the range 0.01-5% w/w, more preferably in an amount in the range 0.1-1.2% w/w, most preferably in an amount in the range 0.3-1% w/w. In a particularly preferred embodiment, the at least one acid is present in an amount in the range 0.5-0.8% w/w.

Preferably, the at least one acid is an organic acid, for example a carboxylic acid, especially a polycarboxylic acid. Examples of suitable acids include but are not restricted to: citric acid, EDTA, oxalic acid, phthalic acid, succinic acid, adipic acid, and lactic acid. A particularly preferred acid is citric acid.

Preferably the at least one acid has a pKa of between 1 and 5, more preferably, between 2 and 4, most preferably about 3.

Preferably the at least one acid is present in an amount sufficient to lower the pH of the sanitizing composition to be in the range 2-5, more preferably 3-4, most preferably, about 3.5.

Preferably, the sanitizing composition further comprises at least one alcohol. Preferably, the at least one alcohol is present in an amount in the range 0.01-5% w/w, more preferably in an amount in the range 0.05-2% w/w, most preferably in an amount in the range 0.15-1% w/w. In a particularly preferred embodiment, the alcohol is present at a concentration in an amount in the range 0.2-0.5% w/w; especially 0.3-0.4% w/w.

Preferably, the at least one alcohol is any Cl to CG, branched or unbranched alcohol. The at least one alcohol may be substituted or unsubstituted. Examples of suitable alcohols include isopropyl alcohol, methanol, ethanol, propan-1-ol, propan-2-ol, butan-1-ol, butan-2-ol, pentan-1ol, pentan-2-ol, pentan-3-ol, hexan-1-ol, hexan-2-ol, hexan-3-ol, cyclohexanol, cyclopentanol, dimethyl butanol. A preferred at least one alcohol is isopropyl alcohol.

When present, chlorine dioxide is preferably present in an amount in the range 0.001-5% w/w, more preferably, in an amount in the range 0.005-1% w/w, most preferably in an amount in the range 0.01-0.5% w/w. In a particularly preferred embodiment chlorine dioxide is present in an amount in the range of 0.02-0.15% w/w, preferably, in an amount in the range 0.03-0.1% w/w.

The sanitizing composition of the first aspect preferably comprises water as balance. The water is preferably present in an amount of at least 82% w/w, preferably at least 90% w/w, and more preferably at least 92% w/w. Most preferably it comprises water in amount of at least 95% w/w. The amount of water present could be up to 100% less the minimum amount of other components, but is preferably up to 99% w/w (the other components preferably being present in an amount of at least 1% in total).

The sanitizing compositions of the first aspect can contain compounds additional to those mentioned above. However preferred compositions of the invention consist essentially of the compounds mentioned above.

A composition of the first aspect may be used neat, to combat pathogens and/or biofilms.

It will be appreciated that components of the composition of the first aspect may be supplied by two different chemical compounds. For example there may be two antimicrobial agents (as stated expressly above); two non-ionic surfactants; and so on. When this happens the definitions of the amounts of a particular component given above refer to the total complement of that component, even though that complement is supplied by two (or more) chemical compounds. The same comment applies to definitions given later in relation to further embodiments of the invention.

According to a second aspect of the present invention there is provided a concentrate composition able to be diluted by water to produce a composition capable of destroying pathogens present at a locus and/or of removing biofilm present at a locus, the concentrate composition comprising, in aqueous solution:

at least one surfactant, preferably non-ionic, preferably present in an amount in the range 1-20% w/w;

at least one antimicrobial agent, preferably present in an amount in the range 0.05-15% w/w;

at least one acid, preferably present in an amount in the range 1-25%;

and, optionally, chlorine dioxide or source thereof, preferably present in an amount in the range 0.1-15% w/w.

Preferably, the chemical nature of the surfactant in the second embodiment, is as defined above in relation to the first aspect.

Preferably the at least one surfactant is present, in the second embodiment, in an amount in the range 2-15% w/w, more preferably in an amount in the range 3-10% w/w, most preferably in an amount in the range 4-8% w/w. In a particularly preferred embodiment, the at least one surfactant is present in an amount in the range 5-6% w/w.

Preferably, the chemical nature of the at least one antimicrobial agent, in the second embodiment, is as defined above in relation to the first aspect.

Preferably, the at least one antimicrobial agent is present, in the second embodiment, in an amount in the range 0.1-12% w/w, more preferably in an amount in the range 0.5-10% w/w, most preferably in an amount in the range 1-7% w/w. In a particularly preferred embodiment, the at least one antimicrobial agent is present in an amount in the range 2-5% w/w, preferably in an amount in the range 3-4% w/w.

The antimicrobial agent may comprise a quaternary ammonium compound. Preferably, the chemical nature of the quaternary ammonium compound, in the second embodiment, is as defined above in relation to the first aspect.

When a quaternary ammonium antimicrobial agent is present, it is preferably present in an amount in an amount in the range 0.01-10% w/w, more preferably in an amount in the range 0.05-8% w/w, more preferably in an amount in the range 0.1-6% w/w, and most preferably in an amount in the range 0.5-4% w/w; and especially, in an amount in the range 2-3% w/w.

The antimicrobial compound may comprise a biguanide compound. Preferably, the chemical nature of the biguanide compound, in the second embodiment, is as defined above in relation to the first aspect.

When a biguanide antimicrobial agent is present, it is preferably present in an amount in an amount in the range 0.01-6% w/w, more preferably in an amount in the range 0.05-4% w/w, more preferably in an amount in the range 0.1-3% w/w, and most preferably in an amount in the range 0.5-2% w/w; and especially, in an amount in the range 1-1.5% w/w.

Preferably, the chemical nature of the at least one acid, in the second embodiment, is as defined above in relation to the first aspect.

Preferably the at least one acid is present, in the second embodiment, in an amount in the range 3-50% w/w, more preferably in an amount in the range 3-45% w/w, most preferably in an amount in the range 4-40% w/w. In a particularly preferred embodiment, the at least one acid is present in an amount in the range 30-40% w/w, preferably, in an amount in the range 35-40% w/w.

Preferably the at least one acid is present in an amount, in the second embodiment, sufficient to lower the pH of the concentrate composition to between 2 and 5, more preferably between 2.5 and 4, most preferably, about 3. Preferably the chlorine dioxide or source thereof and the acid are introduced to each other, to form the composition of the second embodiment, shortly before use.

Preferably, the concentrate composition of the second embodiment further comprises at least one alcohol.

Preferably, the chemical nature of the at least one alcohol, in the second embodiment, is as defined above in relation to the first aspect.

Preferably, the at least one alcohol is present in the second embodiment, in an amount in an amount in the range 0.5-12% w/w, more preferably in an amount in the range 1-10% w/w, most preferably in an amount in the range 2-8% w/w. In a particularly preferred embodiment, the alcohol is present at a concentration of about 4% w/w.

Preferably, the chemical nature of the chlorine dioxide or source thereof, in the second embodiment, is as defined above in relation to the first aspect.

When present in the second embodiment, chlorine dioxide is preferably present in an amount, or equivalent amount when it is in stabilised form, in the range 0.05-10% w/w, more preferably, in an amount in the range 0.1-8% w/w, most preferably in an amount in the range 0.2-6% w/w. In a particularly preferred embodiment chlorine dioxide is present in an amount in the range of 0.5-4% w/w, preferably, in an amount in the range 1-3% w/w.

In a preferred embodiment, the concentrate composition is produced from a plurality of precursor compositions; preferably, as two precursor compositions.

In such an embodiment, the chlorine dioxide is provided separate from the at least one acid and may be kept in an alkaline solution. Under alkaline conditions, chlorine dioxide (ClO₂) undergoes reduction to chlorite (ClO₂⁻). In this manner chlorine dioxide is stabilized in solution.

However, as one would expect, upon acidification of the stabilized chlorine dioxide solution, the chlorite is transformed back to chorine dioxide. Hence chlorine dioxide gas is liberated.

Preferably the concentrate composition of the second aspect yields, on dilution, the composition in accordance with the first aspect. Preferably the dilution ratio (concentrate composition:additional water) is in the range 1:1-1:100, more preferably 1:4-1:50, and still more preferably 1:8-1:25. Most preferably the dilution ratio is in the range 1:10-1:15; especially preferred is a ratio of 1:13.

Further preferred concentration definitions applicable to the concentrate composition of the second aspect may be derived by taking any of the concentration definitions given above in relation to the first aspect, and multiplying them by the number 13.

Further preferred concentration definitions applicable to the composition of the first aspect may be derived by taking any of the concentration definitions given above in relation to the second aspect, and dividing them by the number 13.

According to a third aspect of the present invention there is provided a concentrate composition of the second aspect, formed by mixing first and second precursor compositions, wherein:

the first precursor composition comprises, at least one acid;

and the second precursor composition comprises an aqueous solution of stabilised chlorine dioxide.

Preferably the at least one acid according to the third aspect is present in an amount sufficient to render the pH of the respective first precursor composition to between 1.8 and 4.8, more preferably between 2.8 and 4, most preferably, between 3.2 and 3.8.

Preferably, according to the third aspect, the pH of the second precursor composition is in the range 7.5 to 11, more preferably in the range 8 to 10, most preferably about 8.5.

Preferably the at least one surfactant and the at least one antimicrobial agent may be in the first or in the second precursor composition, or they may be partitioned between both precursor compositions. Preferably they are both in the first precursor composition.

The components of the first and second precursor compositions of the third aspect are suitably of chemical nature defined above in relation to the first aspect.

The components of the first and second precursor compositions of the third aspect are present in such amounts, and the first and second precursor compositions are mixed in such proportions, that in relation to the resulting concentrate composition the numerical definitions given in relation to the second aspect are satisfied.

The definitions given above as regards the preferred amounts of the components of the second aspect apply to corresponding components of the first precursor compositions of the third aspect, when multiplied by a factor of 1.5.

The definitions given above as regards the preferred amount of the component(s) of the second aspect apply to the corresponding component(s) of the third aspect, when multiplied by a factor of 3.

The definitions given above as regards the preferred amounts of the components of the first aspect apply to corresponding components of the first precursor compositions of the third aspect, when multiplied by a factor of 17.

The definitions given above as regards the preferred amount of the component(s) of the first aspect apply to the corresponding component(s) of the third aspect, when multiplied by a factor of 33.

The first or second precursor compositions may be used in their own right (without the second precursor composition), in combating pathogens and/or biofilm.

According to a fourth aspect of the present invention there is provided a sanitizing kit comprising separate containers of first and second precursor compositions of the third aspect.

According to a fifth aspect of the present invention there is provided a method of preparing a concentrate composition of the second aspect comprising mixing the first precursor composition as defined in the third aspect with the second precursor composition of the third aspect.

Preferably, according to the fifth aspect, the first precursor composition is added to the second precursor composition in a ratio between 10:1 and 0.1:1, more preferably between 5:1 and 0.5:1, most preferably between about 3:1 and 1:1. In a particular preferred embodiment, the first precursor composition is added to the second precursor composition in a ratio between 1:1 and 2:1. Most preferably it is between 1.8:1 and 2.2:1.

Preferably, after adding the first precursor composition to the second precursor composition shortly before use, an activation time is allowed.

By activation time it is meant, a suitable time for the acid of one of the two precursor compositions to destabilize the stabilized chlorine dioxide of the other of the two precursor compositions. The length of this period of time may vary depending on factors such as; the volume of precursor compositions mixed, the temperature at which they are mixed, whether the mixture is agitated and the pKa of the acid etc.

Once the activation time has elapsed, the resultant mixture is then preferably diluted, preferably in water, to form the sanitizing composition of the first aspect. Preferably, the resultant mixture is added to the diluent in a ratio between 1:1 and 0.001:1, more preferably in a ratio between 0.5:1 and 0.005:1, most preferably in a ratio between 0.1:1 and 0.01:1. In a particularly preferred embodiment, the ratio of the resultant mixture to the diluent is approximately 0.08:1.

According to a sixth aspect of the present invention there is provided use of a sanitizing composition of the first aspect to kill or disable pathogens.

According to an seventh aspect of the present invention there is provided use of a sanitizing composition of the first aspect to destroy or degrade biofilm.

According to a eighth aspect of the present invention, there is provided use of a sanitizing composition of the first aspect both to kill or disable pathogens and to destroy or degrade biofilm.

According to a ninth aspect of the present invention there is provided use of a precursor compound of the third aspect, also containing at least one non-ionic surfactant and at least one antimicrobial agent, to kill or degrade pathogens and/or to destroy or degrade biofilm.

The “pathogens” of the sixth, eighth and ninth aspects are preferably bacteria, and in particular MRSA and/or Legionella. By “disable” we mean that if the pathogens are not destroyed their essential functioning is disrupted; especially their ability to reproduce and/or grow.

The above invention will now be illustrated by reference to the following examples.

### EXAMPLE 1

The ability of a sanitizing composition according to the present invention (Composition 1) to neutralise canine parvovirus (CPV) was tested with and without faecal material in order to simulate clean and dirty conditions.

Testing was carried out as follows;

Composition 1 was prepared by the addition of 30 ml of liquid Component A and 30 ml of liquid Component B to 1 litre of distilled water. The solution was stirred together for five minutes then added to 1 litre of water.

### Component A

| | |
|---|---|
| Water | 43% |
| Citric acid | 40% |
| Non-ionic surfactant | 6.5% |
| Fragrance | 0.5% |
| Biguanide (PHMB) | 2% |
| Quaternary ammonium | 4% |
| Compound (BAC) | |
| Isopropyl alcohol | 4% |

### Component B

Stabilised chlorine dioxide 2% solution.

Virus was mixed with and without faeces and with and without Composition 1 to give the test material and controls required. Table 1 presents the composition of each control and test solution.

TABLE 1
Composition of controls and test solutions

| | 1 | 2 | 3 | 4 |
|---|---|---|---|---|
| CPV | 1 mL | 1 mL | 900 μL | 900 μL |
| Composition 1 | 1 mL | None | 1 mL | None |
| Faeces | None | None | 0.1 g | 0.1 g |
| Media | None | 1 mL | None | 1 mL |

For testing under clean conditions, the CPV virus was thawed under cold running water and an equal volume of Composition 1 or cell maintenance media (“Media” herein) added. The composition was mixed thoroughly by rotating. Timing began as soon as Composition 1 was added.

For testing under dirty conditions, 0.1 g of normal dog faeces was weighed in to a vessel and 1 mL of CPV virus thawed under cold running water. 900 μL of CPV was added to the faeces and the vessel gently shaken until the faecal sample was in suspension. 1 mL of Composition 1 or media was added and mixed by rotating the vessel. Timing began as soon as the Composition 1 was added. All reactions were carried out at room temperature.

Samples were collected from each solution at 1, 2, 3, 5, 10, 20, 30 and 60 minute time intervals. At each time interval, a sample was taken and immediately diluted 1 in 10 in media by adding 200 μL of the solution to 1800 μL of media. The vials were kept on ice. At the end of the hour, the dirty samples were filtered to reduce toxicity to the cell culture and each diluted sample was further diluted by 7 serial 10-fold steps in the following manner: 7 micro centrifuge tubes containing 900 μL of media were prepared in advance, 100 μL of the 1 in 10 dilution of the test solution or control was taken and pipetted into 900 μL of media. The tip was changed, the solution aspirated 8 times and 100 μL transferred to the next microcentrifuge tube. This was repeated for each tube in the series.

The control and samples were assessed for the presence of virus by inoculating 50 μL of solution into microtitre plates. Each dilution step was added in quadruplicate and then 100 μL of CRFK cell suspension (conc 3×10⁵) was dispensed into each well and the plates incubated at 37° C. for four days.

After 4 days incubation, the plates were fixed, stained and examined for specific fluorescence. The clean solution containing the disinfectant composition was toxic at the 10⁻¹ dilution. A summary of the results is shown in Table 2.

TABLE 2
Titres of CPV (Log₁₀TCID₅₀/50 μL) in samples taken
from solutions at defined time intervals

| Time (mins) | Solution 1 | Solution 2 | Solution 3 | Solution 4 |
|---|---|---|---|---|
| 1 | ND | 3.75 | 2.50 | 4.00 |
| 2 | ND | 3.75 | 2.50 | 3.50 |
| 3 | ND | 3.75 | 2.25 | 3.50 |
| 5 | ND | 3.50 | 2.50 | 3.25 |
| 10 | ND | 2.75 | 2.50 | 3.50 |
| 20 | ND | 3.75 | 2.50 | 3.25 |
| 30 | ND | 3.00 | 3.25 | 3.50 |
| 60 | ND | 3.25 | 2.75 | 4.50 |

ND = No virus detected (≦1.5)

The preparations contained an appropriate initial concentration of parvovirus. Solutions 2 and 4 record the thermo stability of the virus at room temperature both in culture medium and when diluted with dog faeces (solution 4). Over the 60 minute period of observation there was no significant reduction in viral titre.

Solutions 1 and 3 record the effect of Composition 1. No virus was detected under the clean conditions where virus was mixed with the Composition 1 although the 1st dilution of the test was unreadable due to cell toxicity.

### Conclusion

Composition 1 formulation appears to have a significant effect on canine parvovirus under the test conditions. Composition 1 caused at least a 100 fold reduction on virus titre under the clean test conditions. Under dirty test conditions some virus was detected in the Composition 1 treated solution, but it would appear that there was an approximate 10 fold reduction in viral titre from the control solution.

For Composition 1, significant reduction in titre was observed and it can be concluded that Composition 1 has a good disinfectant effect. The effect seen was rapid (within one minute of addition), and was not increased by further incubation.

### EXAMPLE 2

### Test Organisms:

| | |
|---|---|
| Staphylococcus aureus | NCTC 10788 |
| Pseudomonas aeruginosa | NCTC 6749 |
| Escherichia coli | NCTC 10418 |
| Enterococcus hirae | NCTC 12367 |
| Methicillin resistant | NCTC 12493 |
| Staphylococcus aureus | |

### Test Methods and Validation:

EN 1276 Chemical disinfectants and antiseptics—Quantitative suspension test for the evaluation of bactericidal activity of chemical disinfectants and antiseptics used in food, industrial, domestic and institutional areas (Phase 2, step 1).

### Requirement:

The test product when tested in accordance with the test methodology described under simulated clean and dirty conditions shall demonstrate at least a 5 log 10 reduction.

Product diluent used during the test Sterile hard water 300 mg/kg CaCO₃

### Product Test Concentration:

Undiluted Composition 2 was prepared by mixing 50 ml Precursor Composition C and 25 ml Precursor Composition D. Precursor Composition C comprised:
600 parts by weight of water;
80 parts by weight SYNPERONIC N (a ethoxylated nonylphenol non-ionic surfactant, from ICI);
200 parts of a citric acid solution that is 50% w/w citric acid in water.
20 parts by weight of PHMB (polyhexamethylbiguanide)
40 parts by weight of BAC (benzyl ammonium chloride)
60 parts by weight of propan-2-ol.

Precursor Composition D comprised a 20,000 ppm solution of chlorine dioxide stabilised by being in alkaline solution (pH 8.5).

Precursor Compositions C (60 ml) and Precursor Composition D (30 ml) were mixed and left for 5 mins, then added to 1 litre of water.

### Test Conditions:

| | |
|---|---|
| Appearance product dilution | Pale blue solution |
| Contact time | 30 sec, 1, 2 and 5 minutes |
| Test temperature | 20° C. |
| Interfering substance: | Bovine albumin |
| | 0.03% clean conditions |
| | 0.03% dirty conditions |
| Inhibition method: | Dilution/neutralization |
| Neutralizer: | Tween 80 40 g/1, sodium |
| | lauryl sulphate |
| | 10 g/I, lecithin 4 g/1, |
| | sodium thiosulphate |
| | 5 g/l, saponin 30 g/litre. |

Tests were performed to establish the suitability of this neutralizer in neutralizing the activity of the disinfectant without being inhibitory to the test organisms. Initial tests were carried out with the standard neutralizer described in EN 1276 but this proved unsatisfactory as a neutralizer. An increase in the Tween 80 and lecithin concentration and the addition of Sodium lauryl sulphate was required.

### Summary of Test Methods:

EN 1276 Chemical disinfectants and antiseptics—Quantitative suspension test for the evaluation of bactericidal activity of chemical disinfectants and antiseptics used in food, industrial, domestic and institutional areas (Phase 2, step 1).

Copies available from BSI, 389 Chiswick High Road, London W4 4AL.

The test method involves mixing 1 ml of the test bacteria with 1 ml of soil (0.3 or 3% albumin and then adding 8 ml of disinfectant. After the required contact time, 1 ml is removed to 9 ml of recovery/neutralizer, which is then plated to detect surviving test bacteria.

### Results

Bactericidal activity of Composition 2 using phase 2 step 1 suspension test EN 1276

Log₁₀ counts/reductions achieved in 30 secs, 1, 2 and 5 minutes (Tests carried out in duplicate)

Log₁₀ reduction

| | Log₁₀ | | | | | | | | |
|---|---|---|---|---|---|---|---|---|---|
| | initial | Clean conditions | | | | Dirty conditions | | | |
| Test | count | (0.03% albumin) | | | | (0.03% albumin) | | | |
| organism | (challenge) | 30 sec | 1 min | 2 min | 5 min | 30 sec | 1 min | 2 min | 5 min |
| P. aerug. | 7.00 | <6.00 | <6.00 | <6.00 | <6.00 | <6.00 | <6.00 | <6.00 | <6.00 |
| E. coli | 7.00 | <6.00< | <6.00 | <6.00 | <6.00 | <6.00 | <6.00 | <6.00 | <6.00 |
| E. Hirae | 6.15 | <5.15 | <5.15 | <5.15 | <5.15 | <5.15 | <5.15 | <5.15 | <5.15 |
| S. aureus | 6.85 | <5.85 | <5.85 | <5.85 | <5.85 | <5.85 | <5.85 | <5.85 | <5.85 |
| MRSA | 6.04 | <6.04 | <6.04 | <6.04 | <6.04 | <6.04 | <6.04 | <6.04 | <6.04 |

A contact time of 5 mins is stated in EN 1276. However 30 sec, 1, 2 and 5 mins contact times were employed, since the shorter times were felt to be more relevant for a hard surface disinfectant for use in health care premises. The tests carried out using a shorter contact time were therefore more stringent than as described in EN 1276.

### CONCLUSION

When tested in accordance with EN 1276 (1997), undiluted Composition 2 solution possesses bactericidal activity at 20° C. A >5 Log₁₀ (99.999%) reduction was achieved with all test organisms i.e. Ps. aeruginosa, Staph. aureus, Esch. coli, Ent. ltirae and MRSA in 30 secs, 1 min, 2 mins and 5 mins under clean (0.03% albumin) and dirty (0.3% albumin) conditions. To satisfy the requirements for the test, at least a 5 Log₁₀ reduction in specified test organisms is required within 5 mins when the disinfectant is tested at its intended use dilution(s). Composition 2, therefore, satisfies the requirements of the test.

Performance under light (clean) and moderate to heavy (dirty) soiling was assessed. The presence of soil does not appear to affect the performance of the product.

It is believed that a sanitizing composition made in accordance with the present invention effectively and efficiently destroys bacteria and removes biofilm. A sanitizing composition in accordance with the present invention may be used in any situation where the spread of bacteria is unwanted, or the removal of biofilm is required. One such environment is in medical facilities.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A sanitizing composition capable of destroying pathogens present at a locus and/or of removing biofilm present at a locus, the composition comprising, in aqueous solution:
at least one surfactant;
at least one antimicrobial agent;
at least one acid;
and, optionally chlorine dioxide.

2. A sanitizing composition as claimed in claim 1, wherein the antimicrobial agent comprises a quaternary ammonium compound.

3. A sanitizing composition as claimed in claim 1, wherein the antimicrobial agent comprises a biguanide compound.

4. A sanitizing composition as claimed in claim 1, wherein the pH of the composition is in the range 2-5.

5. A sanitizing composition as claimed in claim 1, and containing at least one alcohol.

6. A sanitizing composition as claimed in claim 1, wherein said at least one surfactant comprises at least one non-ionic surfactant.

7. A concentrate composition able to be diluted with water to produce a sanitizing composition as claimed in any preceding claim, the concentrate composition comprising, in aqueous solution:
at least one surfactant;
at least one antimicrobial agent;
at least one acid;
and, optionally, chlorine dioxide or source thereof.

8. A concentrate composition as claimed in claim 7 and containing chlorine dioxide or a source thereof, wherein the concentrate composition is provided as two precursor compositions, one containing the chlorine dioxide or source thereof under alkaline conditions, and the other containing the acid; the mixing thereof yielding an acidic composition from which chlorine dioxide is liberated.

9. A concentrate composition as claimed in claim 7, formed by mixing first and second precursor compositions, wherein:
the first precursor composition comprises at least one acid;
and the second precursor composition comprises an aqueous solution of stabilised chlorine dioxide.

10. A sanitizing kit comprising separate containers of first and second precursor compositions as claimed in claim 9.

11. A method of preparing a sanitizing composition as claimed in claim 1, the method comprising:
mixing a first precursor composition with a second precursor composition thereby forming a concentrate composition; allowing a dwell time; and, at the expiry of the dwell time, diluting the concentrate composition to form the sanitizing composition.

12. Use of a sanitizing composition as claimed in to kill or disable pathogens.

13. Use of a sanitizing composition as claimed in claim 1 to destroy or degrade biofilm.

14. Use of a sanitizing composition as claimed in claim 1 both to kill or disable pathogens and destroy or degrade biofilm.

15. Use of a first precursor compound as defined in claim 9, and containing at least one surfactant and at least one antimicrobial agent, to kill or disable pathogens and/or destroy or degrade biofilm.

16. (canceled)
